SEQUENCE LISTING (15 sequences)

SEQ ID NO: 1 - DNA, 68 bp, Artificial Sequence
  Synthetic - Reference nucleotide shown in Figure 2
agatttatcgcttctgtgacagacagtgaaaacacaaatcaaagagaagctgcaagtcatggtaagtc

SEQ ID NO: 2 - protein, 5 aa, Artificial Sequence
  Synthetic - First reading frame shown in Figure 2, part 1
DLSLL

SEQ ID NO: 3 - protein, 16 aa, Artificial Sequence
  Synthetic - First reading frame shown in Figure 2, part 2
QTVKTQIKEKLQVMVS

SEQ ID NO: 4 - protein, 8 aa, Artificial Sequence
  Synthetic - Second reading frame shown in Figure 2, part 1
IYRFCDRQ

SEQ ID NO: 5 - protein, 11 aa, Artificial Sequence
  Synthetic - Second reading frame shown in Figure 2, part 2
KHKSKRSCKSW

SEQ ID NO: 6 - protein, 23 aa, Artificial Sequence
  Synthetic - Third reading frame shown in Figure 2
RFIASVTDSENTNQREAASHGKS

SEQ ID NO: 7 - protein, 21 aa, Artificial Sequence
  Synthetic - Reference protein shown in Figure 2
RFIASVTDSENTNQREAASHG

SEQ ID NO: 8 - protein, 15 aa, Artificial Sequence
  Synthetic - Amino acid sequence shown in Figure 3B
VTDSENTNQREAASH

SEQ ID NO: 9 - DNA, 45 bp, Artificial Sequence
  Synthetic - Nucleotide sequence shown in Figure 3B
gtgacagacagtgaaaacacaaatcaaagagaagctgcaagtcat

SEQ ID NO: 10 - protein, 15 aa, Artificial Sequence
  Synthetic - Amino acid sequence shown in Figure 3C
VTDSENTNQRSCKSW

SEQ ID NO: 11 - DNA, 45 bp, Artificial Sequence
  Synthetic - Nucleotide sequence shown in Figure 3C
gtgacagacagtgaaaacacaaatcaaagaagctgcaagtcatgg

SEQ ID NO: 12 - protein, 12 aa, Artificial Sequence
  Synthetic - Amino acid sequence shown in Figure 3D
VTDSEIKEAASH

SEQ ID NO: 13 - DNA, 36 bp, Artificial Sequence
  Synthetic - Nucleotide sequence shown in Figure 3D
gtgacagacagtgaaatcaaagaagctgcaagtcat

SEQ ID NO: 14 - protein, 13 aa, Artificial Sequence
  Synthetic - Amino acid sequence shown in Figure 3E
VTVKTQIKEAASH

SEQ ID NO: 15 - DNA, 39 bp, Artificial Sequence
  Synthetic - Nucleotide sequence shown in Figure 3E
gtgacagtgaaaacacaaatcaaagaagctgcaagtcat

(19) United States
(12) Patent Application Publication
(10) Pub. No.: US 2022/0267860 A1
(43) Pub. Date: Aug. 25, 2022

(54) LONGITUDINAL MOLECULAR DIAGNOSTICS DETECT SOMATIC REVERSION MUTATIONS

(71) Applicant: Tempus Labs, Inc., Chicago, IL (US)

(72) Inventor: Shelly Marie Sorrells, Chicago, IL (US)

(21) Appl. No.: 17/651,793

(22) Filed: Feb. 19, 2022

Related U.S. Application Data
(60) Provisional application No. 63/151,398, filed on Feb. 19, 2021.

(51) Int. Cl. C12Q 1/6886

(52) U.S. Cl. CPC C12Q 1/6886; C12Q 2600/156; C12Q 2600/158

(57) ABSTRACT

The present disclosure provides methods for treating a subject that has been diagnosed with cancer. The methods utilize longitudinal genomic testing to monitor the progression of a subject's cancer over time. Specifically, the methods involve comparing sequencing data collected from paired tumor-normal samples and liquid biopsies to sequencing data collected from the same sample types at an earlier time point to identify changes in the tumor genomic profile.

### CROSS-REFERENCE TO RELATED APPLICATIONS

This application claims priority to U.S. Provisional Application No. 63/151,398 filed on Feb. 19, 2021, the contents of which are incorporated by reference in their entireties.

### STATEMENT REGARDING FEDERALLY SPONSORED RESEARCH

N/R.

### SEQUENCE LISTING

A Sequence Listing accompanies this application and is submitted as an ASCII text file of the sequence listing named “166619_00180_ST25.txt” which is 4,153 bytes in size and was created on Dec. 29, 2021. The sequence listing is electronically submitted via EFS-Web with the application and is incorporated herein by reference in its entirety.

### BACKGROUND

Drug resistance is a central problem in cancer treatment. While many cancer cells are initially responsive to a treatment, they can evolve to evade the treatment. There are a variety of different biological mechanisms that result in drug resistance, including DNA mutations that change the function of proteins and pathways within the cell. In some cases, acquired drug resistance arises after a reversion of an oncogenic mutation that restores the wild-type phenotype to a tumor cell.

Longitudinal cancer profiling using matched tumor-normal biopsies can be used to monitor genomic changes, including reversion mutations, in solid tumors. However, such analyses are limited to the study of identified solid tumors that are accessible for biopsy, and they fail to provide information regarding potential metastases and actionable mutations that were not present in the primary tumor. Accordingly, there remains a need in the art for methods that detect the evolution of mutations in tumor cells over the entire course of cancer treatment, both within known tumors and throughout the body.

### SUMMARY

The present disclosure provides methods of treating a subject that has been diagnosed with cancer. The methods comprise: (a) at a first time point (1) obtaining at least three biological samples from the subject, wherein at least one of the samples comprises a solid tumor sample, wherein at least one of the samples comprises a matched normal sample, and wherein at least one of the samples comprises a blood plasma sample; (2) isolating nucleic acid from each sample; (3) sequencing the nucleic acid from each of the samples to obtain genetic sequence information; (4) comparing the sequence information obtained in step 3 to a wild-type reference sequence for the species of the subject to identify mutations; and (5) treating the subject with a first cancer treatment based on the comparison made in step 4; (b) at a second time point, repeating steps 1-3; (c) comparing the sequence information obtained for the solid tumor sample in step b3 at the second time point with the sequence information obtained for the solid tumor sample in step a3 at the first time point, and comparing the sequence information obtained for the blood plasma sample in step b3 at the second time point with the sequence information obtained for the blood plasma sample in step a3 at the first time point to identify changes in the sequencing information; and (d) treating the subject with a second cancer treatment based on the comparison made in step c. In various embodiments, the sequence information obtained for the solid tumor sample in step b3 at the second time point may be compared with the sequence information obtained for the blood plasma sample in step a3 at the first time point and/or the sequence information obtained for the blood plasma sample in step b3 at the second time point may be compared with the sequence information obtained for the solid tumor sample in step a3 at the first time point. In one example, sequence information may include a detection status (presence or absence status) and/or a quantitative measure (for example, variant allele fraction/VAF or estimated circulating tumor fraction) associated with each variant in a group of selected variants.

### BRIEF DESCRIPTION OF THE DRAWINGS

FIG. 1 is a timeline of the patient procedures, treatments, and disease progression for the case study disclosed in Example 1. Green boxes denote genomic testing, purple boxes denote treatments, and pink boxes denote clinical time points and diagnostic testing.

FIG. 2 shows the evolution of the BRCA2 alterations observed over the course of the case study disclosed in Example 1. The alterations are depicted as an Integrative Genomics Viewer (IGV) visualization of BRCA2 sequencing data. (A, B) Genomic analysis of whole blood and tumor tissue from a bone metastasis reveals a two-base pair (bp) deletion in both, indicating a germline alteration. (C) Genomic analysis of a metastatic liver lesion reveals both the original two bp deletion, as well as an additional seven bp deletion, resulting in an in-frame somatic reversion. (D, E) Genomic analysis of circulating tumor DNA (ctDNA) from blood plasma shows the previously identified germline alteration and somatic reversion alteration, as well as a secondary somatic reversion alteration. The horizontal pink and blue bars represent individual reads in the forward and reverse strand sequence orientation, respectively. The grey histogram indicates the relative sequencing coverage at each individual nucleotide position. A decrease in coverage is expected at the location of the deletions. Nucleotide deletions are represented as short horizontal black bars with the size of the deletion specified below the sequencing reads. A reference nucleotide sequence (SEQ ID NO: 1) and reference protein sequence (SEQ ID NO: 7) are included at the bottom of the figure. The three possible open reading frames (ORFs) (ORF 1 is provided as SEQ ID NOs: 2 and 3, ORF 2 is provided as SEQ ID NOs: 4 and 5, and ORF 3 is provided as SEQ ID NO: 6) are shown in grey, with the third being the wild-type reading frame.

FIG. 3 shows a schematic depiction of multiple BRCA2 reversion alterations that restore the open reading frame interrupted by the germline mutation. (A) A BRCA2 protein diagram illustrating the domains of this protein. (B) The wild-type nucleotide (SEQ ID NO: 9) and amino acid (SEQ ID NO: 8) sequences corresponding to amino acids 250-264 of the BRCA2 protein, which is the region in which the germline and somatic alterations were detected. (C) A two base pair deletion causes a frameshift in BRCA2 in the germline. (D) A somatic deletion of seven base pairs causes a reversion mutation that restores the open reading frame of the germline alteration. (E) A four base pair deletion causes a second somatic reversion mutation that also restores the open reading frame of the germline alteration.

FIG. 4 is a flow diagram of exemplary methods disclosed herein.

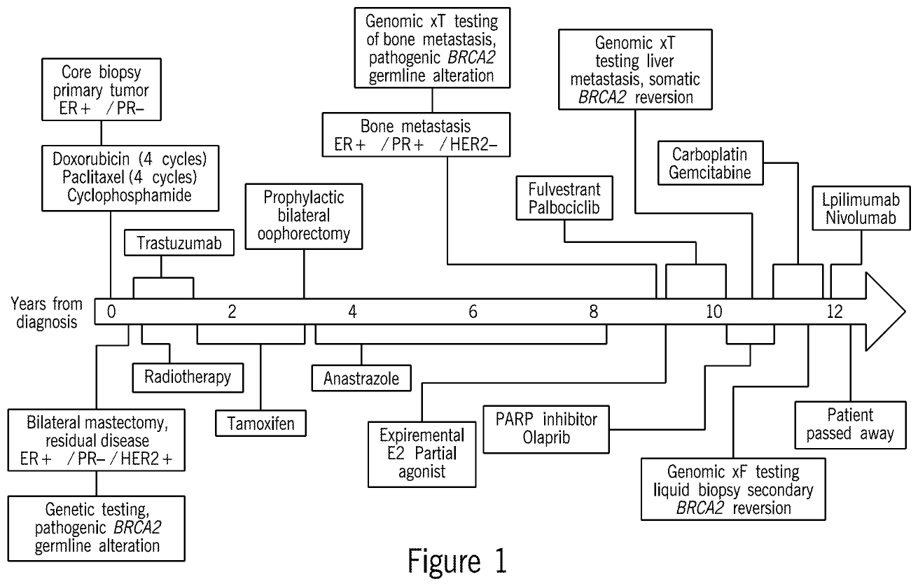

### DETAILED DESCRIPTION

The present application is directed to methods of treating cancer that utilize longitudinal genomic testing to study the progression of the cancer over time. These methods pair tumor-normal sampling with liquid biopsies to detect actionable mutations in both the primary tumor and in metastases, allowing treatment decisions to be based on the subject's current tumor genomic profile.

Reversion mutations are a major cause of acquired resistance to cancer therapeutics. Thus, one of the primary goals of the disclosed methods is to detect reversion mutations that develop over the course of a subject's cancer treatment. Some of the best-studied examples of such reversions include those that involve a mutation in the BRCA1 or BRCA2 gene. A tumor cell may initially form due to an oncogenic alteration in BRCA1 or BRCA2, which may be either germline or somatic in origin. However, under the selective pressure of a cancer treatment, such tumors can develop reversion mutations that revert the previously inactivated BRCA1 or BRCA2 gene into a functional gene, thus allowing the tumor to evade cell death and become resistant to the treatment. Due to the limited number of targeted therapies for tumor suppressors, BRCA1 and BRCA2 are the primary examples of genes that develop reversion mutations. However, as more targeted treatments are developed, other examples will become prevalent.

Patients with pathogenic germline BRCA1 or BRCA2 mutations have an increased risk of breast, ovarian, pancreatic, prostate, and other cancers. Tumors that arise in these patients typically exhibit loss-of-heterozygosity (LOH) or epigenetic silencing in the wild-type BRCA allele, resulting in the production of truncated BRCA protein and defective homologous recombination DNA repair¹. This homologous recombination deficiency (HRD) renders DNA particularly vulnerable to damage caused by double-strand breaks, resulting in an accumulation of mutations over time and increased carcinogenesis². However, HRD also renders BRCA1/2-mutant cancers sensitive to DNA-damaging treatments, such as radiation^{3,4}, platinum-based therapies^{5,6}, and poly ADP-ribose polymerase (PARP) inhibitors^{7,8}.

PARP inhibitors target the highly abundant proteins PARP1 and PARP2, which play an important role in transcription, chromatin modification, and DNA repair⁹. As a result, PARP inhibition targets DNA repair through multiple mechanisms of action, including PARP trapping^{10,11,} inhibition of base excision repair of single strand breaks⁸, and indirect activation of non-homologous end-joining^{12-14.} In tumors with HRD, such as those with BRCA alterations, PARP inhibition is especially effective because multiple DNA repair pathways are simultaneously impaired, resulting in synthetic lethality⁶⁻⁸.

In BRCA-mutant breast cancers, single-agent PARP inhibitor treatment induces partial response rates in as high as 47% of patients, and complete responses lasting 60 weeks in up to 33% of patients¹⁵⁻¹⁷. Recent studies suggest that response rates continue to improve with combined treatment regimens. However, despite its initial effectiveness, BRCA-mutant cancers often develop resistance to PARP inhibition^{18,19}. While many potential mechanisms for this resistance have been described, BRCA reversion mutations have emerged as a key resistance mechanism and have been described in a number of recent cases²⁰⁻²³ BRCA reversions occur when acquired somatic mutations, typically insertions/deletions (indels) or base substitutions, restore the open reading frame of the altered BRCA allele, allowing it to produce a functional protein that restores efficient homologous recombination DNA repair. As a result, PARP inhibition no longer causes synthetic lethality, leading to drug resistance and disease progression.

This application is based, at least in part, on the present inventors' study of a patient with pathogenic germline BRCA2-driven breast cancer that acquired resistance to the PARP inhibitor olaparib (see Examples). This clinical resistance was likely the result of an acquired somatic reversion mutation, which was detected using a matched tumor-normal genomic analysis. A second reversion mutation was later detected via genetic sequencing of circulating tumor DNA (ctDNA) in blood plasma following carboplatin treatment, indicating a likely new site of metastasis and source of resistance. This case study highlights the benefits of performing comprehensive genomic testing throughout the course of disease to track the evolution of tumor mutations.

### Methods:

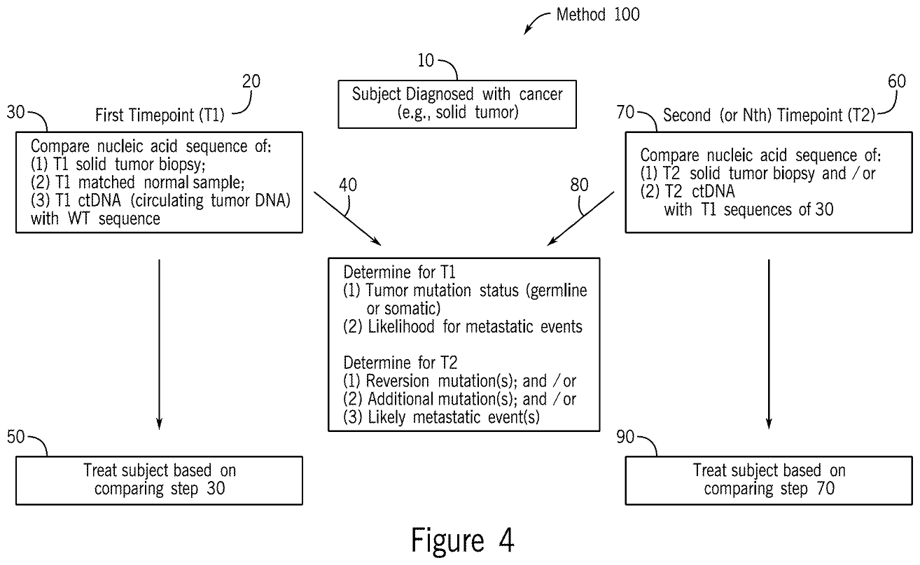

The present disclosure provides methods of treating a subject that has been diagnosed with cancer. Referring to FIG. 4, one embodiment of the methods disclosed herein comprises method 100: (a) at a first time point 20, (1) obtaining at least three biological samples from the subject 10, wherein at least one of the samples comprises a solid tumor sample, wherein at least one of the samples comprises a matched normal sample, and wherein at least one of the samples comprises a blood plasma sample; (2) isolating nucleic acid from each sample; (3) sequencing the nucleic acid from each of the samples to obtain genetic sequence information; (4) comparing the sequence information obtained in step 3 to a wild-type reference sequence for the species of the subject to identify mutations 30; and (5) treating the subject with a first cancer treatment 50 based on the comparison made in step 4; (b) at a second time point 60, obtaining from the subject at least one biological sample selected from a solid tumor sample, a blood plasma sample, or both a solid tumor sample and a blood plasma sample, and repeating steps 2-3; (c) comparing the sequence information obtained in step b3 at the second time point with the sequence information obtained in step a3 at the first time point 70; and (d) treating the subject with a second cancer treatment based on the comparison made in step c 90.

The methods of the present disclosure provide several advantages over the prior art. The combination of longitudinal nucleic acid sequencing of tumor tissue and liquid biopsy samples provides the ability to follow the evolution of mutations in tumor samples, to identify metastatic events (FIG. 4 at 40, 80), and to more quickly and efficiently determine whether to pursue or withdraw a course of treatment based on an analysis of both the liquid and solid tumor samples at a given time (FIG. 4 at 50, 90). The use of a combination of sample types (solid tumor biopsy, normal tissue, and liquid biopsy, 30) can provide a more holistic view of the different tumor subclones within the patient, and the tumor-normal matched sequencing data can be used to reveal the tissue origin of any genetic alterations.

The methods of the present disclosure involve collecting sequence information for at least three distinct biological samples: a solid tumor sample, a matched normal sample, and a blood plasma sample. The term “biological sample” refers to a sample taken from the subject. The biological samples may be fresh, frozen, or formalin fixed paraffin embedded (FFPE) samples. In various embodiments, for each time point, only one or two of the biological samples described here is collected and available for processing to obtain sequence information. In other embodiments, for one time point, at least these three biological samples are collected.

The term “solid tumor sample” refers to a biopsy collected from the solid tumor itself. Solid tumor samples include, but are not limited to, specimens collected from the tumor using a fine needle, core needle, or incisional biopsy of the tumor, or excisions, resections, and cell blocks from cytology specimens.

In the methods disclosed herein, the solid tumor sample is paired with a matched normal sample at the first time point, forming a pair of samples referred to as a “matched tumor-normal sample”. The term “matched normal sample” refers to a sample that was collected from healthy tissue in the same individual. The matched normal sample may be collected using the same methods that are used to collect the solid tumor sample. The matched normal sample may also be collected as a saliva sample or as a peripheral blood draw. A comparison between the sequence information derived from the solid tumor sample to that of the matched normal sample is used, for example, to determine whether a detected mutation is a germline mutation or a somatic mutation (FIG. 4 at 40).

The terms “germline mutation,” “germline variant,” and “germline alteration” are used interchangeably herein to refer to a change in the DNA of a gamete. Because gametes give rise to all the cells that make up an organism, germline alterations are passed on to every cell in the body. Cancer caused by germline alterations is referred to as inherited or hereditary cancer. In contrast, a “somatic mutation,” “somatic variant,” “somatic alteration,” or “acquired mutation” is a mutation that arose in a single somatic cell in the body and is only passed on to cells and tissues derived from that cell. Understanding whether an alteration is a germline or somatic alteration is important, as the identification of a germline alteration can help to characterize the cancer risk of a subject and inform the need for additional surveillance. Further, the detection of a germline alteration can lead to the identification of family members that share the alteration and are therefore predisposed to developing a malignancy. Understanding one's predisposition to cancer allows one to take preventative measures to decrease cancer risk.

The solid tumor sample obtained at the second time point may be from the same solid tumor that was sampled at the first time point. Re-sampling of the same tumor can be used to identify any new somatic mutations that this tumor has acquired. Alternatively, the solid tumor sample obtained at the second time point may be from a different solid tumor sample obtained at the first time point. For example, the solid tumor sampled at the second time point may be a tumor that was more recently detected than the tumor sampled at the first time point.

The third sample used with the present methods is a blood plasma sample. A blood plasma sample may be collected, for example, by drawing whole blood from the subject, centrifuging the blood (for at least 15 minutes at 2200-2500 RPM), and moving the separated plasma to a new vial. In cancer patients, the blood plasma contains DNA and RNA that is released from tumor cells into the bloodstream when tumor cells undergo apoptosis, necrosis, or exosome excretion. The DNA released from the tumor comprises short DNA fragments (approximately 166 bp in length), which are referred to as “circulating tumor DNA (ctDNA)”. Thus, in some embodiments, the nucleic acid isolated from the blood plasma sample comprises circulating tumor DNA. ctDNA is one form of “cell-free DNA (cfDNA)”, a broader term which describes DNA that is freely circulating in the bloodstream but is not necessarily of tumor origin. ctDNA is obtained from a liquid biopsy. A “liquid biopsy” is a blood sample taken from a patient to monitor tumor progression. Liquid biopsies from the peripheral blood are less invasive than solid tumor biopsies, and can be used in circumstances in which a traditional solid tumor biopsy is not possible (for example, because the tumor is not accessible or the patient is too ill to undergo the procedure). Importantly, in cases in which a metastatic tumor comprises a different mutation than the known solid tumor, liquid biopsies enable the detection of metastasis and mutations that may lead to drug resistance.

After sequencing has been repeated at the second time point, the sequence information obtained at the first time point and the second time point are compared (FIG. 4 at 70) to identify any changes in the tumor genetic profile that have occurred between the first and second time point. The sequence information obtained from any of the samples may be compared. For example, sequence information obtained for a solid tumor sample at the second time point may be compared to sequence information obtained from the solid tumor sample, the blood plasma sample, or both the solid tumor sample and blood plasma sample at the first time point. Likewise, sequence information obtained for a blood plasma sample at the second time point may be compared to sequence information obtained from the solid tumor sample, the blood plasma sample, or both the solid tumor sample and blood plasma sample at the first time point. In some embodiments, one or more of the samples at the second time point is compared to the matched normal sample from the first time point. In some embodiments, any sequence information obtained for a solid tumor sample at the second time point is compared only to the sequence information obtained for the solid tumor sample at the first time point, and any sequence information obtained for a blood plasma sample at the second time point is compared only to the sequence information obtained for the blood plasma sample at the first time point.

A second cancer treatment is selected for the subject based on this comparison (FIG. 4 at 90). The second cancer treatment may involve continuation of the current treatment, discontinuation of the current treatment, addition of a treatment, or a change of treatment (e.g., if one or more of revision mutations, additional mutations, or metastatic events are identified, FIG. 4 at 80). In some embodiments, additional time points are included in the method. For example, in some embodiments, the sequence information obtained at a third time point is compared to the sequence information obtained at the second time point, and a third cancer treatment is selected based on this comparison. In some embodiments, at least 2, at least 3, at least 4, at least 5, at least 6, at least 7, at least 8, at least 9, or at least 10 time points are included in the method. In some embodiments, a time point other than the first time point is referred to as the “Nth” timepoint.

Time points may be days, weeks, months, or years apart. For example, an Nth time point may be coordinated with a course of treatment (for example, scheduling a time point immediately before and immediately after a course of treatment), recommended based on patient symptoms (for example, patient response to a first therapy, side effects, exacerbation of symptoms, new symptoms, tumor size), or at intervals selected by a physician based on experience and patient need.

As used herein, the terms “sequence information” or “genetic sequence information” refer to the nucleotide sequences of the nucleic acids in the biological samples. Sequence information may be obtained using any sequencing method. Sequence information may include DNA, RNA, or a combination of DNA and RNA sequences. In the present methods, sequence information is analyzed to identify clinically relevant oncogenic mutations. As used herein, the terms “genetic mutation”, “mutation”, “genetic alteration”, and “alternation” are used interchangeably to refer to a permanent change in a gene sequence. Mutations include base pair substitutions, insertions, deletions, copy number alterations, and rearrangements. The sequence information obtained in the present methods can be used (1) to identify oncogenic mutations, and (2) to characterize the mutations as somatic mutations or germline mutations. Oncogenic mutations may be identified by comparing the sequence information obtained from the biological samples to a wild-type reference sequence for the species of the subject. As used herein a “wild-type reference sequence” is a gene sequence that is considered “normal” or free of oncogenic mutations. A wild-type reference sequence may be identified, for example, by sequencing a gene of interest in a subject or a cohort of subjects that are cancer free. This reference sequence may also be (or be derived from) the standard reference sequence GRCh37 (hg19) from the Genome Reference Consortium, GRCh38 Genome Reference Consortium Human Build 38, or a subsequently standardized genome reference sequence. A mutation may be characterized as a somatic or germline mutation by comparing the sequence information from a tumor sample to that of the matched normal sample. Any conclusions related to the sequence information may be reported to a clinician who is responsible for the subject's medical care.

In some embodiments, a mutation that was not detected at the first time point is detected at the second time point in the solid tumor sample, in the blood plasma sample, or in both the solid tumor sample and the blood plasma sample *FIG. 4 at 80). In these embodiments, the second cancer treatment should be selected in view of the newly discovered mutation (FIG. 4 at 90). For example, if a new mutation is discovered in the solid tumor sample, the second cancer treatment may comprise a drug that has shown clinical activity in cancers comprising that mutation (FIG. 4 at 90).

In some embodiments, a reversion mutation is detected at the second time point in the solid tumor sample, in the blood plasma sample, or in both the solid tumor sample and the blood plasma sample (FIG. 4 at 90). As used herein, the term “reversion mutation” refers to a second mutation in a gene that restores gene function that was lost as a result of a first mutation in that gene (for example, by restoring the open reading frame). Several types of genetic mutations can cause reversions, including missense mutations, insertions or deletions that cause frameshift mutations, or in-frame insertions or deletions. Restoration of gene activity by a reversion mutation can underlie the development of resistance to a therapy. For example, reversion mutations that restore BRCA2 activity can cause resistance to therapies targeting cells deficient in DNA damage repair, such as PARP inhibitors. Thus, in these embodiments, the second cancer treatment is selected in view of a reversion mutation. For example, if a reversion mutation is discovered in the solid tumor sample and the first cancer treatment comprises a drug that targets that mutation, a different drug should be selected for the second cancer treatment (FIG. 4 at 90). However, if the reversion mutation is discovered in the blood plasma sample but not in the solid tumor sample, it may be reasonable to continue treatment with the drug that targets that mutation while increasing surveillance because clinical relapse is likely.

In some embodiments, the sequencing information obtained from the solid tumor and the blood plasma sample is the same at the first time point but is different at the second time point. In these embodiments, the clinician may decide to evaluate the subject for metastases, as these results indicate that the tumor may have evolved or metastasized (FIG. 4 at 80). Metastases may be detected using blood tests, tumor marker tests, and/or imaging methods (such as an ultrasound, CT scan, bone scan, MM, or PET scan).

In some cases, comparing the sequence information collected at the two time points will allow clinicians to change or adjust the treatment strategy to be more effective for the treatment of the subject. For instance, the cancer treatment may be adjusted to add an additional therapeutic or to remove a particular therapeutic. In the Examples, the inventors describe a case study of a subject with a BRCA2-driven breast cancer. The subject was initially prescribed a PARP inhibitor, as these drugs have shown efficacy for the treatment of BRCA-mutant breast cancers (see Background). However, two independent BRCA2 reversion mutations were detected in metastasized tumors at later time points, and treatment with the PARP inhibitor was discontinued to the benefit of the patient. Thus, in some embodiments, the first cancer treatment is discontinued based on the comparison, and the second cancer treatment is different from the first cancer treatment. In other embodiments, the first and second cancer treatments may be the same.

In the present methods, nucleic acids isolated from the biological samples are sequenced at two time points: a first time point and a second time point. While these time points may be taken at any stage of disease progression, it will likely be advantageous to take the first time point soon after the subject has been diagnosed with cancer such that the initial cancer treatment can be tailored to the subject's unique tumor genomic profile. The second time point would ideally be taken at a stage in which sequencing information could aid in a treatment decision. In some embodiments, the second time point is taken after disease progression occurs. For example, the second time point may be taken after the cancer has relapsed, metastasized, or developed resistance to the first cancer treatment. In other embodiments, the second time point is taken at or near the end of the first cancer treatment.

As used herein, the term “tumor genomic profile” refers to the genetic makeup of a subject's tumor(s). In cases in which a subject has multiple tumors that comprise distinct mutations, this term is used to describe the genetic profile of all the subject's tumors collectively. A tumor genomic profile may also be specified for a particular tumor. The tumor genomic profile can be determined using various assays including, but not limited to, next generation sequencing and digital droplet PCR (ddPCR).

In the Examples, the inventors describe a case study in which the patient has a BRCA2-driven breast cancer. However, the methods of the present disclosure may be used to monitor the evolution of any oncogenic mutation. As used herein, the term “oncogenic mutation” is used to describe any genetic mutation that promotes the development of cancer, and it includes both germline and somatic mutations. Exemplary oncogenic mutations include, without limitation, a mutation in the gene APC, ATM, AXIN2, BMPRIA, BRCA1, BRCA2, BRIP1, CDCl73, CDH1, CDK4, CDKN2A, CEBPA, CHEK2, DKC1, EGFR, EPCAM ETV6, FH, FLCN, GATA2, GREM1, KIT, MAX, MEN1, MET, MLH1, MSH2, MSH3, MSH6, MUTYH, NBN, NF1, NF2, NTHL1, PALB2, PDGFRA, PMS2, POLD1, POLE, PRKAR1A, PTCH1, PTEN, RAD51C, RAD51D, RB1, RET, RUNX1, SCG5, SDHAF2, SDHB, SDHC, SDHD, SMAD4, STK11, TERC, TINF2, TP53, TSC1, TSC2, VHL, or WT1. Oncogenic mutations of interest include mutations in tumor suppressor genes (including tumor suppressor genes likely to be associated with reversion mutations, and/or targeted by therapies in developmental stages or approved therapies, such as BRCA1, BRCA2, ATM, BARD1, BRIP1, CDK12, CHEK1, CHEK2, EZH2, FANCL, NF1, PALB2, PTCH1, RAD51B, RAD51C, RAD51D, RAD54L, SMARCB1, TSC1, TSC2, TP53, PTEN, CDH1, CDKN2A, and CDKN2B) and mutations in oncogenes (such as EGFR, ERBB2 (HER2) and the RAS family genes). In various embodiments, the protein products of these genes are the target of one or more therapies.

In some embodiments, an oncogenic mutation is present in the solid tumor initially. In such cases, the mutation may be a driver mutation, that is, a mutation that drives tumorigenesis by conferring certain selective advantages and/or cell cycle disregulation to tumor cells. Exemplary driver mutations include, without limitation, mutations in the BRCA1, BRCA2, ESR1, BRAF, IDH1, IDH2, FGFR1, FGFR2, FGFR3, KIT, or EGFR gene. Such mutations may be detected in the solid tumor sample at the first time point in the methods disclosed herein. In some embodiments, the same mutation that is initially detected in the solid tumor sample is also detected in the matched normal sample at the first time point. This indicates that the mutation is a germline mutation.

The methods of the present disclosure involve isolating and sequencing nucleic acids. As used herein, the terms “nucleic acids”, “polynucleotides”, “oligonucleotides”, and “nucleic acid molecules” are used interchangeably to refer to a polymer of DNA or RNA, which may be single-stranded or double-stranded. The nucleic acids isolated in the present methods may comprise genomic DNA, circulating tumor DNA (ctDNA), circulating tumor RNA (ctRNA), total cellular RNA, or messenger RNA (mRNA).

Nucleic acids may be isolated from the cells or plasma within the biological samples using standard methods that are well known in the art, including those that rely on organic extraction, ethanol precipitation, silica-binding chemistry, cellulose-binding chemistry, and ion exchange chemistry. Many reagents and kits for nucleic acid isolation are commercially available. In some embodiments, nucleic acid isolation may include eliminating DNA molecules from all or a portion of the isolated nucleic acid molecules to isolate only RNA molecules, and/or eliminating RNA molecules from all or a portion of the isolated nucleic acid molecules to isolate only DNA molecules.

The isolated nucleic acids are then used to prepare sequencing libraries. Library preparation may include enrichment for nucleic acid molecules of interest. For example, enrichment may be performed using hybridization capture of specific sequences of interest (for example, an oncology panel). Captured RNA may be reverse transcribed to generate cDNA for sequencing. Library preparation may include adding nucleotide barcodes to the isolated nucleic acid molecule to allow for multiplexing. Library preparation may also include amplifying isolated nucleic acid molecules (for example, using PCR or Illumina bridge amplification).

Any suitable sequencing method may be used with the present methods including, for example, whole genome sequencing, whole-exome sequencing, whole-transcriptome sequencing, single-cell sequencing, and targeted panel sequencing. Thus, the resulting sequencing data may include transcriptional data and/or genomic data associated with one or more genes. The sequencer may provide sequencing data in the form of one or more FASTQ files comprising sequencing reads, and the sequences of the isolated nucleic acids may be determined by analyzing the sequencing reads. In some embodiments, the sequencing is accomplished using a next generation sequencer, such as a NextSeq 550, 10×, Illumina, or another sequencing instrument.

For example, in some embodiments, a whole genome or whole exome sequencing method is used to sequence the nucleic acids in the samples at the first time point. In other embodiments, only the nucleic acids captured by a targeted gene panel (such as the Tempus xT panel or another targeted oncology panel) are sequenced at the first time point. Likewise, in some embodiments, a whole genome sequencing method is used to sequence the nucleic acids in the samples at the second time point, whereas in other embodiments, only the nucleic acids captured by a targeted gene panel are sequenced at the second time point. Thus, in some embodiments, step (c) of the present methods comprises comparing whole genome sequencing results of the solid tumor sample and/or the blood plasma sample obtained at the second time to whole genome sequencing results obtained for these samples at the first time point. Alternatively, in other embodiments, only the sequencing results pertaining to specific genes of interest are compared between the first and second time points. Notably, a more targeted gene comparison may be performed whether the sequencing data comprises whole genome sequencing data or targeted gene panel data. In any case, it will be advantageous to compare the sequencing data pertaining to any oncogenic mutation that was identified at the first time point to monitor for changes such as reversion mutations. All genes with alterations detected at different timepoints are relevant and should be reviewed and are not limited to a single gene or oncogenic alteration. In some embodiments, at least 1, at least 2, at least 3, at least 4, at least 5, at least 6, at least 7, at least 8, at least 9, or at least 10 genes are analyzed using the present methods.

In certain embodiments, the sequence information is obtained using a Tempus xT next-generation sequencing (NGS) assay and/or a Tempus xF NGS assay. The Tempus xT NGS assay is a combined DNA/RNA sequencing method that utilizes tumor-normal matched samples. This method uses a targeted oncology panel for hybrid capture of 595 or 648 genes, depending on the version, and it produces highly accurate somatic alteration calling and whole transcriptome sequencing data (see Beaubier et al., Oncotarget 10(24): 2384-2396, 2019). The Tempus xF NGS assay is a liquid biopsy cell-free DNA assay. This method helps to overcome the low frequency of mutant alleles found in liquid biopsies (due to the high background of wild-type cell-free DNA) by using a targeted oncology panel of 77, 105, or 523 genes, depending on assays.

The present application provides methods for treating cancer in a subject. As used herein the term “cancer,” “cancerous tissue,” or “tumor” refers to an abnormal mass of tissue in which the growth of the mass surpasses and is not coordinated with the growth or death of normal tissue. In the case of hematological cancers, this includes a volume of blood or other bodily fluid containing cancerous cells. A cancer or tumor can be defined as “benign” or “malignant” depending on the following characteristics: degree of cellular differentiation including morphology and functionality, rate of growth, local invasion and metastasis. A “benign tumor” can be well differentiated, have characteristically slower growth than a malignant tumor, and remain localized to the site of origin. In addition, in some cases a benign tumor does not have the capacity to infiltrate, invade, or metastasize to a distant site. A “malignant tumor” can be poorly differentiated (anaplasia) and can have characteristically rapid growth accompanied by progressive infiltration, invasion, and destruction of the surrounding tissue. Furthermore, a malignant tumor can have the capacity to metastasize to distant sites.

Any form of solid tumor may be treated using the methods disclosed herein. Thus, the present methods are not limited to the treatment of the tumor types exemplified in this application (that is, breast cancer, bone cancer, and liver cancer). Exemplary cancer types that can be treated using the present methods include, without limitation, adrenal cancer, basal cell carcinoma, biliary cancer, bladder cancer, bone cancer, brain cancer, breast cancer, cervical cancer, colorectal cancer, endocrine tumor, endometrial cancer, esophageal cancer, gastric cancer, gastrointestinal stromal tumor, glioma, glioblastoma, head and neck cancer, kidney cancer, liver cancer, lymphoma, medulloblastoma, melanoma, meningioma, mesothelioma, neuroblastoma, non-small cell lung cancer, oropharyngeal cancer, ovarian cancer, pancreatic cancer, peritoneal cancer, prostate cancer, renal cell carcinoma, retinoblastoma, sarcoma, skin cancer, small cell lung cancer, testicular cancer, thymoma, thyroid cancer, liquid heme cancers, and solid tumors of unknown origin.

As used herein, “treating” or “treatment” describes the management and care of a subject for the purpose of combating a disease, condition, or disorder. Treating includes administering a treatment to prevent the onset of the symptoms or complications, to alleviate the symptoms or complications, or to eliminate the disease, condition, or disorder. For example, treating cancer in a subject includes the reducing, repressing, delaying, or preventing of cancer growth, reduction of tumor volume, and/or preventing, repressing, delaying, or reducing metastasis of the tumor. Treating cancer in a subject also includes the reduction of the number of tumor cells within the subject.

Any suitable cancer treatment may be prescribed as the first cancer treatment and/or the second cancer treatment in the methods disclosed herein. However, the first and second cancer treatment should each be selected based on the comparison of the sequence information generated in the methods and the type of cancer to be treated. For example, a DNA-damaging treatment (for example, radiation, platinum-based therapies, and PARP inhibitors) may be used to treat a mutation in the BRCA2 gene, while an EGFR inhibitor may be used to treat a copy number variation in the EGFR gene. Exemplary cancer treatments include, without limitation, surgery, radiation, immunotherapies (for example, checkpoint inhibitors and anti-tumor vaccines), targeted therapies (for example, PARP inhibitors and tyrosine kinase inhibitors (TKIs)), stem cell therapies, and hormone therapies. In some embodiments, the first cancer treatment and/or the second cancer treatment comprises a PARP inhibitor. Suitable PARP inhibitors include, without limitation, olaparib (Lynparza), niraparib (Zejula), rucaparib (Rubraca), and talazoparib (Talzenna). In some embodiments, the first cancer treatment and/or the second cancer treatment comprises a platinum-based therapy. Suitable platinum-based therapies include, without limitation, cisplatin, carboplatin, oxaliplatin, nedaplatin, and lobaplatin.

The methods described herein may be particularly useful for responding to cancer progression in subjects that are receiving drugs against which cancers commonly develop resistance. Thus, in some embodiments, the first cancer treatment is a drug against which resistance mechanisms are known. Examples of such drugs include, without limitation, afatinib, alectinib, bosutinib, cabozantinib, capmatinib, cetuximab, crizotinib, dasatinib, entrectinib, erlotinib, gefitinib, ibrutinib, imatinib, larotrectinib, nilotinib, osimertinib, panitumumab, and sunitinib.

As used herein, the term “subject” or “patient” refers to mammals and non-mammals. A “mammal” may be any member of the class Mammalia including, but not limited to, humans, non-human primates (chimpanzees, other apes, and monkey species), farm animals (cattle, horses, sheep, goats, and swine), domestic animals (rabbits, dogs, and cats), or laboratory animals including rodents (rats, mice, and guinea pigs). Examples of non-mammals include, but are not limited to, birds, and the like. The term “subject” does not denote a particular age or sex. In some embodiments, the subject is a human. In some embodiments, the subject has been diagnosed with cancer.

Use with a Digital and Laboratory Health Care Platforms:

The methods described above may be utilized in combination with or as part of a digital and laboratory health care platform that is generally targeted to medical care and research. Many uses of the methods described above, in combination with such a platform, are possible. One example of such a platform is described in U.S. Patent Publication No. 2021/0090694, titled “Data Based Cancer Research and Treatment Systems and Methods”, and published Mar. 25, 2021, which is incorporated herein by reference in its entirety for all purposes.

For example, implementation of the methods may include microservices constituting a digital and laboratory health care platform supporting genetic status tracking and updated therapy matching. Embodiments may include a single microservice for executing and delivering genetic status tracking or may include a plurality of microservices each having a particular role. For example, a first microservice may process nucleic acid sequence data from multiple timepoints to deliver a sequence of genetic statuses over time to a second microservice that matches therapies to the sequence of genetic statuses.

When the methods of the present invention are executed using one or more microservices as part of a digital and laboratory health care platform, the one or more microservices may be part of an order management system that orchestrates the sequence of events to occur at the appropriate time and in the appropriate order. A microservices based order management system is disclosed in U.S. Patent Publication No. 2020/80365232, titled “Adaptive Order Fulfillment and Tracking Methods and Systems”, and published Nov. 19, 2020, which is incorporated herein by reference in its entirety for all purposes.

For example, in embodiments that utilize a first and second microservice, an order management system may notify the first microservice that an order for processing nucleic acid sequence data from multiple timepoints has been received. The first microservice will then execute its function (that is, genetic status tracking) and notify the order management system when its output (the sequence of genetic statuses) is ready for the second microservice. The order management system may then determine that the execution parameters (prerequisites) for the second microservice are satisfied, including that the first microservice has completed its function, and notify the second microservice that it may execute its function (that is, providing a list of matched therapies).

The digital and laboratory health care platform may also include a genetic analyzer system, which may include targeted panels and/or sequencing probes. An example of a targeted panel is disclosed in U.S. Patent Publication No. 2021/0090694, titled “Data Based Cancer Research and Treatment Systems and Methods”, and published Mar. 25, 2021, which is incorporated herein by reference in its entirety for all purposes. Examples of a targeted panel for sequencing cell-free DNA and determining various characteristics of a specimen based on the sequencing is disclosed in U.S. patent application Ser. No. 17/179,086, titled “Methods And Systems For Dynamic Variant Thresholding In A Liquid Biopsy Assay”, filed Feb. 18, 1921; U.S. patent application Ser. No. 17/179,267, titled “Estimation Of Circulating Tumor Fraction Using Off-Target Reads Of Targeted-Panel Sequencing”, filed Feb. 18, 1921; and U.S. patent application Ser. No. 17/179,279, titled “Methods And Systems For Refining Copy Number Variation In A Liquid Biopsy Assay”, filed Feb. 18, 1921; which are incorporated herein by reference in their entirety for all purposes. Targeted panels can be used to deliver next generation sequencing results for genetic status tracking and updated therapy matching. Examples of the design of next-generation sequencing probes is disclosed in U.S. Patent Publication No. 2021/0115511, titled “Systems and Methods for Next Generation Sequencing Uniform Probe Design”, published Jun. 22, 2021 and in U.S. patent application Ser. No. 17/323,986, titled “Systems and Methods for Next Generation Sequencing Uniform Probe Design”, filed May 18, 1921, which are incorporated herein by reference in their entirety for all purposes.

The digital and laboratory health care platform may also include an epigenetic analyzer system. An epigenetic analyzer system analyzes specimens to determine their epigenetic characteristics and may further use that information to monitor a patient over time. An example of an epigenetic analyzer system is disclosed in U.S. patent application Ser. No. 17/352,231, titled “Molecular Response and Progression Detection from Circulating Cell Free DNA”, filed Jun. 18, 1921, which is incorporated herein by reference in its entirety for all purposes.

The digital and laboratory health care platform may also include a bioinformatics pipeline. The bioinformatics pipeline may receive next-generation genetic sequencing results and return a set of binary files, such as one or more BAM files, reflecting DNA and/or RNA read counts aligned to a reference genome. Microservices may then be used to process the DNA and/or RNA read counts, and to produce genetic status tracking and updated therapy matching outputs.

The digital and laboratory health care platform may also include an RNA data normalizer that normalizes any RNA read counts before they are processed by downstream microservices. An example of an RNA data normalizer is disclosed in U.S. Patent Publication No. 2020/0098448, titled “Methods of Normalizing and Correcting RNA Expression Data”, published Mar. 26, 2020, which is incorporated herein by reference in its entirety for all purposes.

The digital and laboratory health care platform may also include a genetic data deconvolver. A genetic data deconvolver is used to deconvolve genetic data generated from a specimen having two or more biological components, allowing one to determine what portion of the genetic data would be associated with each component individually. An example of a genetic data deconvolver is disclosed in U.S. Patent Publication No. 2020/0210852, published Jul. 2, 2020, and PCT/US19/69161, filed Dec. 31, 2019, both titled “Transcriptome Deconvolution of Metastatic Tissue Samples”; and in U.S. patent application Ser. No. 17/074,984, titled “Calculating Cell-type RNA Profiles for Diagnosis and Treatment”, filed Oct. 20, 2020; which are incorporated herein by reference in their entirety for all purposes.

RNA expression levels may be adjusted to be expressed as a value relative to a reference expression level. Furthermore, multiple RNA expression data sets may be adjusted, prepared, and/or combined for analysis, and may be adjusted to avoid artifacts that result from differences in data that were not generated using the same methods, equipment, and/or reagents. An example of RNA data set adjustment, preparation, and/or combination is disclosed in U.S. patent application Ser. No. 17/405,025, titled “Systems and Methods for Homogenization of Disparate Datasets”, filed Aug. 18, 2021, which is incorporated herein by reference in its entirety for all purposes.

The digital and laboratory health care platform may also include an automated RNA expression caller, which compares RNA expression levels associated with multiple samples to determine whether an artifact is causing anomalies in the data. An example of an automated RNA expression caller is disclosed in U.S. Pat. No. 11,043,283, titled “Systems and Methods for Automating RNA Expression Calls in a Cancer Prediction Pipeline”, issued Jun. 22, 2021, which is incorporated herein by reference in its entirety for all purposes.

The digital and laboratory health care platform may also include one or more insight engines that deliver information, characteristics, or determinations related to a disease state that may be based on genetic and/or clinical data associated with a patient, specimen and/or organoid. Exemplary insight engines include a tumor of unknown origin (tumor origin) engine, a human leukocyte antigen (HLA) loss of homozygosity (LOH) engine, a tumor mutational burden engine, a PD-L1 status engine, a homologous recombination deficiency engine, a cellular pathway activation report engine, an immune infiltration engine, a microsatellite instability engine, a pathogen infection status engine, a T cell receptor or B cell receptor profiling engine, a line of therapy engine, a metastatic prediction engine, an TO progression risk prediction engine, and so forth.

An example tumor origin or tumor of unknown origin engine is disclosed in U.S. patent application Ser. No. 15/930,234, titled “Systems and Methods for Multi-Label Cancer Classification”, filed May 12, 1920, which is incorporated herein by reference in its entirety for all purposes.

An example of an HLA LOH engine is disclosed in U.S. Pat. No. 11,081,210, titled “Detection of Human Leukocyte Antigen Class I Loss of Heterozygosity in Solid Tumor Types by NGS DNA Sequencing”, issued Aug. 3, 2021, which is incorporated herein by reference in its entirety for all purposes. An additional example of an HLA LOH engine is disclosed in U.S. patent application Ser. No. 17/304,940, titled “Detection of Human Leukocyte Antigen Loss of Heterozygosity”, filed Jun. 28, 2021, which is incorporated herein by reference in its entirety for all purposes.

An example of a tumor mutational burden (TMB) engine is disclosed in U.S. Patent Publication No. 2020/0258601, titled “Targeted-Panel Tumor Mutational Burden Calculation Systems and Methods”, published Aug. 13, 2020, which is incorporated herein by reference in its entirety for all purposes.

An example of a PD-L1 status engine is disclosed in U.S. Patent Publication No. 2020/0395097, titled “A Pan-Cancer Model to Predict The PD-L1 Status of a Cancer Cell Sample Using RNA Expression Data and Other Patient Data”, published Dec. 17, 2020, which is incorporated herein by reference in its entirety for all purposes. An additional example of a PD-L1 status engine is disclosed in U.S. Pat. No. 10,957,041, titled “Determining Biomarkers from Histopathology Slide Images”, issued Mar. 23, 2021, which is incorporated herein by reference in its entirety for all purposes.

An example of a homologous recombination deficiency engine is disclosed in U.S. Pat. No. 10,975,445, titled “An Integrative Machine-Learning Framework to Predict Homologous Recombination Deficiency”, issued Apr. 13, 2021, which is incorporated herein by reference in its entirety for all purposes. An additional example of a homologous recombination deficiency engine is disclosed in U.S. patent application Ser. No. 17/492,518, titled “Systems and Methods for Predicting Homologous Recombination Deficiency Status of a Specimen”, filed Oct. 1, 2021, which is incorporated herein by reference in its entirety for all purposes.

An example of a cellular pathway activation report engine is disclosed in U.S. Patent Publication No. 2021/0057042, titled “Systems And Methods For Detecting Cellular Pathway Dysregulation In Cancer Specimens”, and published Feb. 25, 2021, which is incorporated herein by reference in its entirety for all purposes.

An example of an immune infiltration engine is disclosed in U.S. Patent Publication No. 2020/0075169, titled “A Multi-Modal Approach to Predicting Immune Infiltration Based on Integrated RNA Expression and Imaging Features”, published Mar. 5, 2020, which is incorporated herein by reference in its entirety for all purposes.

An example of an MSI engine is disclosed in U.S. Patent Publication No. 2020/0118644, titled “Microsatellite Instability Determination System and Related Methods”, published Apr. 16, 2020, which is incorporated herein by reference in its entirety for all purposes. An additional example of an MSI engine is disclosed in U.S. Patent Publication No. 2021/0098078, titled “Systems and Methods for Detecting Microsatellite Instability of a Cancer Using a Liquid Biopsy”, published Apr. 1, 2021, which is incorporated herein by reference in its entirety for all purposes.

An example of a pathogen infection status engine is disclosed in U.S. Pat. No. 11,043,304, titled “Systems and Methods for Using Sequencing Data for Pathogen Detection”, issued Jun. 22, 2021, which is incorporated herein by reference in its entirety for all purposes. Another example of a pathogen infection status engine is disclosed in PCT/US21/18619, titled “Systems and Methods for Detecting Viral DNA From Sequencing”, filed Feb. 18, 2021, which is incorporated herein by reference in its entirety for all purposes.

An example of a T cell receptor or B cell receptor profiling engine is disclosed in U.S. patent application Ser. No. 17/302,030, titled “TCR/BCR Profiling Using Enrichment with Pools of Capture Probes”, filed Apr. 21, 2021, which is incorporated herein by reference in its entirety for all purposes.

An example of a line of therapy engine is disclosed in U.S. Patent Publication No. 2021/0057071, titled “Unsupervised Learning And Prediction Of Lines Of Therapy From High-Dimensional Longitudinal Medications Data”, published Feb. 25, 2021, which is incorporated herein by reference in its entirety for all purposes.

An example of a metastatic prediction engine is disclosed in U.S. Pat. No. 11,145,416, titled “Predicting likelihood and site of metastasis from patient records”, issued Oct. 12, 2021, which is incorporated herein by reference in its entirety for all purposes.

An example of an IO progression risk prediction engine is disclosed in U.S. patent application Ser. No. 17/455,876, titled “Determination of Cytotoxic Gene Signature and Associated Systems and Methods For Response Prediction and Treatment”, filed Nov. 19, 2021, which is incorporated herein by reference in its entirety for all purposes.

The digital and laboratory health care platform may also include a report generation engine that creates a summary report of a patient's genetic profile and the results of one or more insight engines for presentation to a physician. For instance, the report may provide to the physician information about the extent to which the specimen that was sequenced contained tumor or normal tissue. The report may provide a genetic profile for each of the tissue types, tumors, or organs in the specimen. The genetic profile may represent genetic sequences present in the tissue type, tumor, or organ and may include variants, expression levels, information about gene products, or other information that could be derived from genetic analysis of a tissue, tumor, or organ.

The report may also include therapies and/or clinical trials matched based on the genetic profile, insight engine findings, and/or summaries. For example, the therapies may be matched according to the systems and methods disclosed in U.S. patent application Ser. No. 17/546,049, titled “Artificial Intelligence Driven Therapy Curation and Prioritization”, filed Dec. 9, 2021, which is incorporated herein by reference in its entirety for all purposes. The clinical trials may be matched, for example, according to the systems and methods disclosed in U.S. Patent Publication No. 2020/0381087, titled “Systems and Methods of Clinical Trial Evaluation”, published Dec. 3, 2020, which is incorporated herein by reference in its entirety for all purposes.

The report may also include a comparison of the results (for example, molecular and/or clinical patient data) to a database of results from many specimens. An example of methods and systems for comparing results to a database of results are disclosed in U.S. Patent Publication No. 2020/0135303 titled “User Interface, System, And Method For Cohort Analysis”, published Apr. 30, 2020; and in U.S. Patent Publication No. 2020/0211716 titled “A Method and Process for Predicting and Analyzing Patient Cohort Response, Progression and Survival”, published Jul. 2, 2020; which are incorporated herein by reference in their entirety for all purposes. The information may be used, sometimes in conjunction with similar information from additional specimens and/or clinical response information, to match therapies likely to be successful in treating a patient, discover biomarkers, or design a clinical trial.

By way of example, but not by way of limitation, in some embodiments, the methods and systems disclosed herein may further comprises one or more of the following steps: a step for generating a report, and a step for delivering the report to a clinician, e.g., to assist the clinician's decision making process.

Any data generated by the methods and/or the digital and laboratory health care platform may be downloaded by the user. In one example, the data may be downloaded as a CSV file comprising clinical and/or molecular data associated with tests, data structuring, and/or other services ordered by the user. In various embodiments, this may be accomplished by aggregating clinical data in a system backend and making it available via a portal. This data may include variants and RNA expression data, as well as data associated with immunotherapy markers such as MSI and TMB and RNA fusions.

The digital and laboratory health care platform may also include a device comprising a microphone and speaker for receiving audible queries or instructions from a user and delivering answers or other information, such that the methods can be used to add data to a database the device can access. An example of such a device is disclosed in U.S. Patent Publication No. 2020/0335102, titled “Collaborative Artificial Intelligence Method and System”, published Oct. 22, 2020, which is incorporated herein by reference in its entirety for all purposes.

The digital and laboratory health care platform may also include a mobile application for viewing patient records, including genomic sequencing records and/or results. An example of such a mobile application is disclosed in U.S. Pat. No. 10,395,772, titled “Mobile Supplementation, Extraction, and Analysis of Health Records”, issued Aug. 27, 2019, which is incorporated herein by reference in its entirety for all purposes. Another example of such a mobile application is disclosed in U.S. Pat. No. 10,902,952, titled “Mobile Supplementation, Extraction, And Analysis of Health Records”, issued Jan. 26, 2021, which is incorporated herein by reference in its entirety for all purposes. Another example of such a mobile application is disclosed in U.S. Patent Publication No. 2021/0151192, titled “Mobile Supplementation, Extraction, and Analysis of Health Records”, filed May 20, 2021, which is incorporated herein by reference in its entirety for all purposes.

The digital and laboratory health care platform may also include organoids developed in connection with the platform (for example, from the patient specimen), such that the methods can be used to evaluate genetic sequencing data derived from an organoid. Matched therapies may be tested on the organoid, derivatives of that organoid, and/or similar organoids to determine an organoid's sensitivity to those therapies. If the organoid is associated with a patient specimen, any of the results may be included in a report associated with that patient and/or delivered to the patient or patient's clinician. Organoids may be cultured and tested, for example, according to the systems and methods disclosed in U.S. Patent Publication No. 2021/0155989, titled “Tumor Organoid Culture Compositions, Systems, and Methods”, published May 27, 2021; PCT/US20/56930, titled “Systems and Methods for Predicting Therapeutic Sensitivity”, filed Oct. 22, 2020; U.S. Patent Publication No. 2021/0172931, titled “Large Scale Organoid Analysis”, published Jun. 10, 2021; PCT/US2020/063619, titled “Systems and Methods for High Throughput Drug Screening”, filed Dec. 7, 2020; and U.S. patent application Ser. No. 17/301,975, titled “Artificial Fluorescent Image Systems and Methods”, filed Apr. 20, 2021; which are each incorporated herein by reference and in their entirety for all purposes.

The digital and laboratory health care platform may also include an application of one or more of the above functions in combination with or as part of a medical device or a laboratory developed test that is generally targeted to medical care and research, which may be enhanced and personalized through the use of artificial intelligence. An example of laboratory developed tests that are enhanced by artificial intelligence is disclosed in U.S. Patent Publication No. 2021/0118559, titled “Artificial Intelligence Assisted Precision Medicine Enhancements to Standardized Laboratory Diagnostic Testing”, published Apr. 22, 2021, which is incorporated herein by reference in its entirety for all purposes.

It should be understood that the examples provided above are illustrative and do not limit the uses of the methods described herein in combination with a digital and laboratory health care platform.

### Examples

BRCA-mutant cancers can develop therapeutic resistance through several mechanisms. In the following Example, the inventors report a case of pathogenic germline BRCA2-driven breast cancer that was monitored for disease progression and acquired resistance using longitudinal multi-tissue genomic testing. Briefly, genomic testing was performed throughout the course of disease on (1) tumor tissue from multiple sites, (2) circulating tumor DNA from blood plasma, and (3) matched normal tissue. Genomic analyses identified actionable variants for targeted therapies, as well as emerging resistance mutations over time. Specifically, two unique BRCA2 somatic alterations (p.N255fs and p.D252fs) were identified following the development of resistance to PARP inhibitor and platinum treatment, respectively. Both alterations restored the open reading frame of the original germline alteration, likely accounting for the acquired resistance. This case study exemplifies the evolution of multiple subclonal BRCA reversion alterations over time and demonstrates the value of longitudinal multi-tissue genomic testing for monitoring disease progression, predicting measures of response, and evaluating treatment outcomes in oncology patients.

### Methods:

DNA from solid tumor tissue and blood, in addition to circulating tumor DNA (ctDNA) from blood plasma were analyzed by the Tempus xT and xF next-generation sequencing (NGS) assays, respectively (Tempus Labs, Chicago, Ill.). Sequencing was conducted in the Tempus Lab CLIA/CAP-accredited clinical genetics testing laboratory where variant detection, visualization, and reporting were performed as previously described^{32,33}. Data were visualized using Integrative Genomics Viewer³⁴. Written informed patient consent for clinical testing, analysis, and publication was obtained by Tempus Laboratories.

### Case Study:

At the age of 50, a female patient without regular mammography screening presented with a mass in her left breast. Core biopsy of the mass revealed invasive ductal carcinoma that was ER+, PR−, and HER2−(immunohistochemistry [IHC] 1+). Based on the size of the tumor and evidence of lymph node involvement in magnetic resonance imaging (MRI), she received neoadjuvant chemotherapy including four cycles of doxorubicin and cyclophosphamide, followed by four cycles of paclitaxel. The patient then underwent bilateral mastectomy. Pathologic analysis of the left breast revealed 2.5 cm of residual malignancy, which was again found to be ER+, PR−. HER2 was 2+(IHC) with a HER2 ratio at 2.5 based on fluorescence in situ hybridization (FISH). At this time, the patient started adjuvant tamoxifen treatment and completed a one-year course of trastuzumab without complication. Germline genetic testing revealed a pathogenic BRCA2 germline alteration, and the patient opted for a bilateral oophorectomy two years after starting tamoxifen. Her anti-estrogen therapy was changed to anastrozole, which she maintained for an additional five years. The patient palpated a mass in her right axilla, but imaging workup did not show definitive evidence of malignancy or a target for biopsy (mammogram, breast ultrasound, magnetic resonance imaging [Mill], and Positron Emission Tomography/Computed Tomography [PET/CT]).

One year after cessation of anti-estrogen therapy and negative imaging workup, the patient developed pain in her right breast chest wall. A chest CT identified a lesion on the sternum and a subsequent PET/CT revealed numerous bone metastases. A dominant lesion on the sternum was biopsied and revealed ductal carcinoma that was ER+, PR+ and HER2 1+(IHC, FISH, respectively).

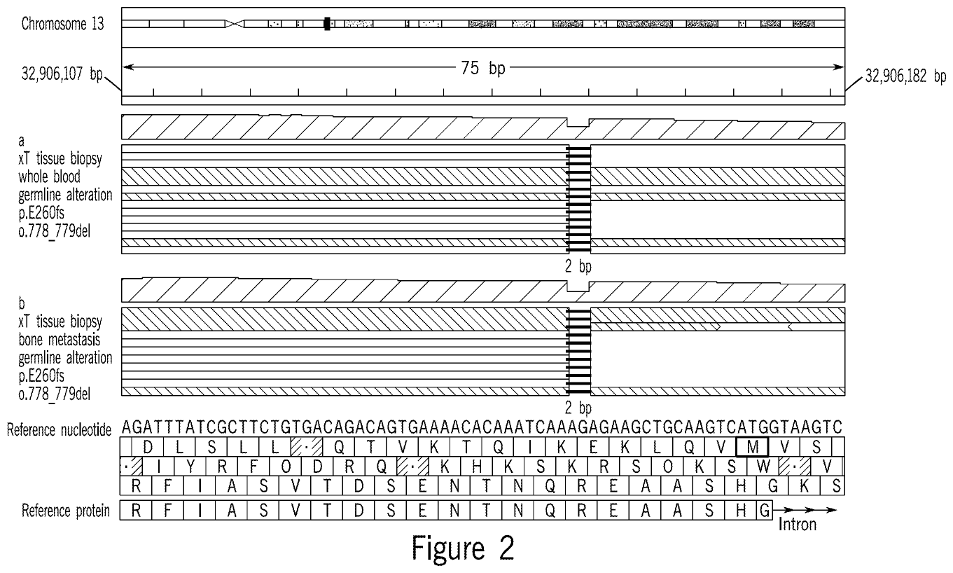

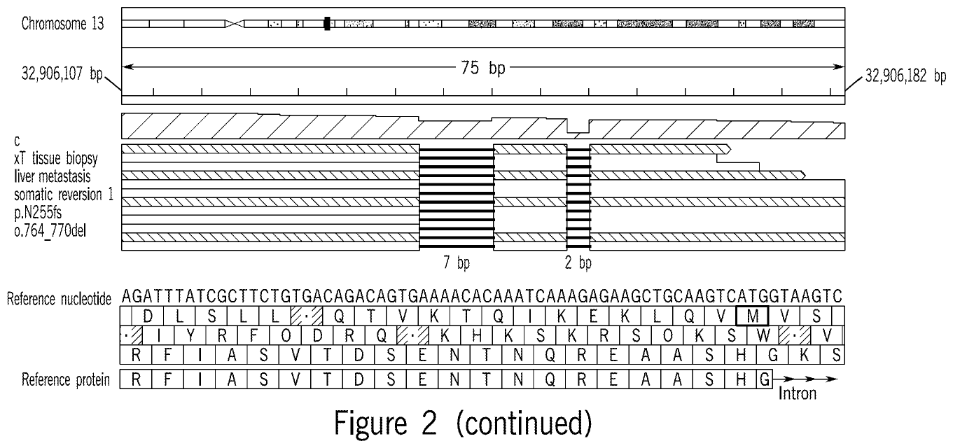

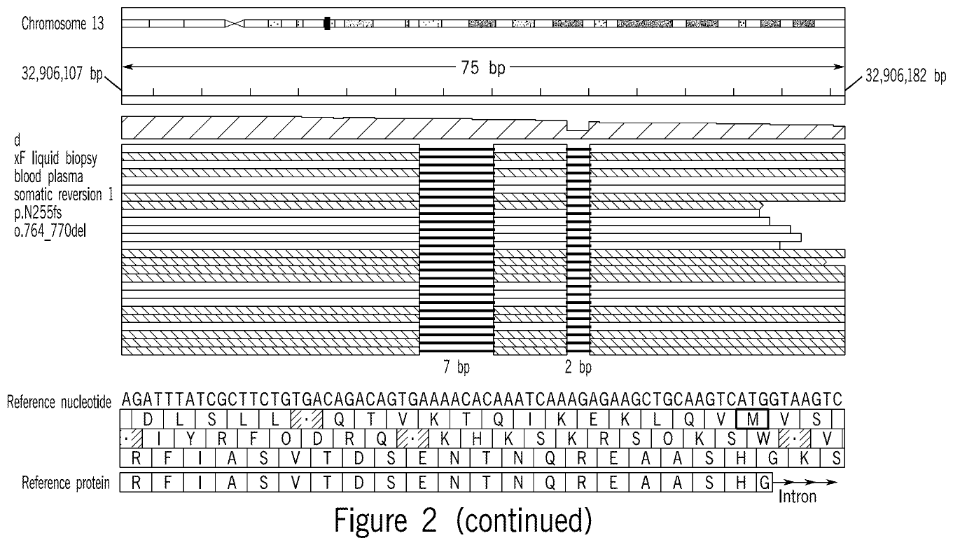

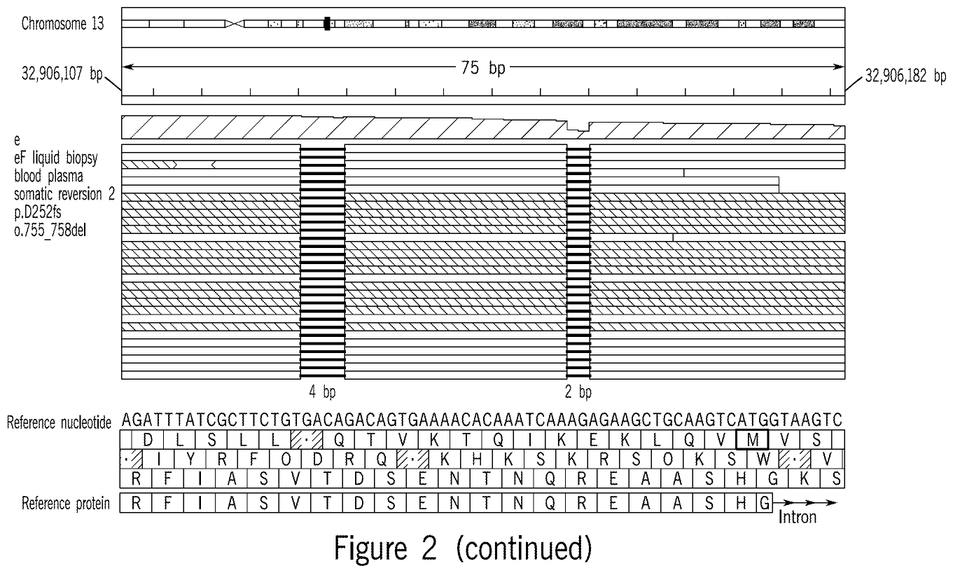

At the time of the initial cancer diagnosis, next generation sequencing (NGS) was not part of a typical clinical workup. However, the metastatic disease was diagnosed 10 years after the initial diagnosis, and in view of the evidence of clinical utility and a change in hormone status, the metastatic lesion was sent for NGS sequencing. The matched tumor-normal genomic analysis of the metastatic bone lesion and blood sample confirmed the presence of the known germline BRCA2 alteration (p.E260fs, c.778_779del, ClinVar variation ID 38119, FIGS. 2A,B). Somatic loss-of-heterozygosity in BRCA2 was not detected in the sequencing results, suggesting that the metastatic bone lesion did not harbor the reversion mutation that developed in the original tumor. However, in addition to the germline BRCA2 alteration, copy number gains in CDK4 and MYC were also identified, which are known to be oncogenic. The patient was treated with fulvestrant (an estrogen receptor antagonist) and palbociclib (a CDK4 inhibitor) for one year, at which time she developed progression. She was briefly treated with an experimental estrogen partial agonist, but progressed shortly after. At this point, the patient began treatment with PARP inhibitor olaparib, based on the germline BRCA2 alteration.

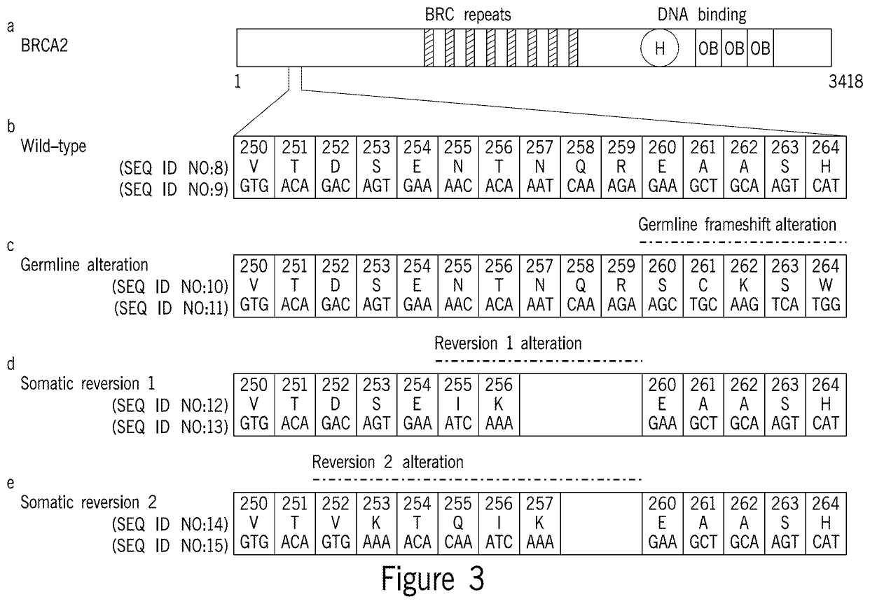

While the patient initially responded well to PARP inhibition, she developed liver metastases after nine months. Genomic analysis of a metastatic liver lesion revealed both the original BRCA2 germline alteration and a somatic reversion alteration. The somatic alteration was in cis (on the same allele) with the pathogenic germline alteration and was detected in BRCA2 at a variant allele frequency (VAF) of 18.1%. This somatic alteration (p.N255fs, c.764_770del) resulted in an in-frame indel and subsequent restoration of the BRCA2 reading frame (p.N255_R259delinsIK, c.764_776delinsTCAA), likely accounting for the resistance to PARP inhibition (FIGS. 2C, 3). The patient was then started on carboplatin and gemcitabine with excellent response in liver metastases and continued on maintenance therapy.

A liquid biopsy from blood plasma was obtained five months later for ctDNA sequencing. The genomic analysis identified the known somatic and germline BRCA2 alterations (1% and 53.4% VAF, respectively), as well as an additional unique somatic BRCA2 alteration (p.D252fs, c.755_758del) at 0.9% VAF. The secondary somatic BRCA2 mutation was also in cis with the pathogenic germline alteration, but was in trans with the first somatic reversion mutation. The second somatic mutation resulted in an in-frame indel and thus represents a second subclonal reversion mutation (FIGS. 2D-E, 3). Additionally, the liquid biopsy revealed pathogenic variants in ESR1 (p.Y537S, c.1610A>C) and TP53 (p.G266V, c.797G>T). Due to elevated tumor mutational burden (TMB) in the patient's first sequencing results, and the possibility that previous therapies may have contributed to the development of neoantigens, the patient briefly underwent immunotherapy with one cycle of ipilimumab and nivolumab therapy. However, liver function worsened so therapy was discontinued. Shortly after discontinuation, the patient passed away at the age of 63.

### Discussion:

In this Example, we report a case study in which longitudinal diagnostic testing using multiple assays and tissue types was used to track cancer progression. The patient acquired resistance to PARP inhibitor olaparib as a result of a somatic BRCA2 reversion mutation that restored the open reading frame of a germline frameshift alteration. This case is consistent with recent reports of BRCA reversions in both germline and somatic alterations identified in prostate cancer, ovarian cancer, and breast cancer after treatment with PARP inhibitors, which were associated with resistance to treatment^{20-22,24,25.} However, in most studies to date, the timing and mechanism of the reversion alterations remain unclear due to a lack of longitudinal testing that started before the reversion alteration appeared. Indeed, many patients receive different lines of treatment or neoadjuvant therapies before PARP inhibitor treatment that may induce the accumulation of mutations^{26,27}. It is possible that a small clonal population of cells with a reversion mutation could exist in a patient for years, and once PARP inhibition is initiated, those cells are selected for and become the dominant clone.

This case exemplifies how multiple subclonal BRCA reversion alterations can develop over time and it highlights the utility of combined tumor/normal/blood biopsies in routine care of cancer patients. For example, genomic analysis of matched tumor-normal samples enables a more thorough understanding of germline and somatic alterations and can identify co-existing actionable variants that may have been overlooked in standard genetic tests. In addition to the previously identified germline BRCA alteration in this case, CDK4 and MYC copy number alterations were identified by NGS of matched tumor-normal tissue, informing subsequent treatment decisions. Indeed, CDK4 inhibition with palcociclib allowed for a year of successful treatment before treatment with PARP inhibitor olaparib began.

Like many patients with BRCA-mutant cancers, the patient of the present study initially responded favorably to PARP inhibition. However, after nine months of PARP inhibitor therapy, the patient was diagnosed with progressive disease and metastasis to the liver. Genomic analysis of the liver metastasis revealed a somatic reversion mutation absent from the previous bone metastasis biopsy. This somatic reversion restored the open reading frame in tumor cells, enabling the synthesis of an in-frame BRCA2 protein and efficient DNA repair through homologous recombination, which is consistent with the resistance to PARP inhibitor treatment this patient experienced.

Additionally, this patient received a liquid biopsy several months later that identified a second reversion alteration. It is unclear whether the second subclonal mutation was present in another tumor site when the first reversion was identified, if it was acquired after discontinuation of PARP inhibition, or if it was acquired in response to the platinum treatment, as has been previously documented^{24,28}. Genomic solid tissue analysis is reliable for identifying driver alterations in solid tumors. However, due to the heterogeneous nature of tumors and emerging resistance mutations, this analysis is somewhat limited for detecting the diversity of mutations associated with advanced cancers. Indeed, many studies have identified actionable mutations in metastatic lesions that were not present in the primary tumors²⁹⁻³¹. Because tumor cells from multiple locations can shed DNA into the blood, liquid biopsies can detect alterations present in distant metastases. As such, analysis of circulating tumor DNA (ctDNA) from liquid biopsies is increasingly used in combination with tissue analyses. In the case of the patient presented here, in addition to the second subclonal reversion, genomic analysis of ctDNA also revealed actionable mutations in ESR1 and TP53 that were not present in either of the two solid tissue analyses. We think that ctDNA analysis will be especially beneficial for identifying early resistance to PARP inhibitors before disease progression is detected by other tests, allowing patients to switch to a more effective treatment for their specific cancers.

Critical questions remain in the treatment of BRCA-mutant cancers. For example, for which patients should serial genomic testing be performed, and how should reversion alterations affect decision-making? While all patients would ideally have access to serial genomic testing, this is unlikely to be feasible in the near future due to costs and availability. However, serial testing is particularly relevant for patients taking drugs with known resistance mechanisms, such as PARP inhibitors and platinum-based therapies, or other targeted cancer therapeutics, including inhibitors of various gene products. How reversion alterations may affect clinical decision-making likely depends on whether the reversion was detected in the tumor tissue or in blood plasma (liquid biopsy). For example, if a reversion alteration is detected through liquid biopsy, but not in the tumor tissue biopsy, continuation of PARP inhibitor treatment may be reasonable. However, such a patient would benefit from being more closely monitored, as the identification of the reversion alteration in ctDNA indicates imminent progression.

To summarize, in this case study, serial NGS sequencing with multiple assays and sample types, including paired solid tumor/normal and liquid biopsy, revealed the evolution of BRCA reversions (i.e., the genetic source of drug resistance) as well as additional actionable variants for targeted therapy. This demonstrates the value of routine genomic testing in clinical care of oncology patients for monitoring disease progression, predicting measures of response, and evaluating treatment outcomes.

### REFERENCES

1 Taniguchi, T. et al. Disruption of the Fanconi anemia-BRCA pathway in cisplatin-sensitive ovarian tumors. Nature medicine 9, 568-574, doi:10.1038/nm852 (2003).
2 Roy, R., Chun, J. & Powell, S. N. BRCA1 and BRCA2: different roles in a common pathway of genome protection. Nat Rev Cancer 12, 68-78, doi:10.1038/nrc3181 (2011).
3 Grosse, N. et al. Deficiency in homologous recombination renders Mammalian cells more sensitive to proton versus photon irradiation. Int J Radiat Oncol Biol Phys 88, 175-181, doi:10.1016/j.ijrobp.2013.09.041 (2014).
4 Baert, A. et al. Analysis of chromosomal radiosensitivity of healthy BRCA2 mutation carriers and non-carriers in BRCA families with the G2 micronucleus assay. Oncol Rep 37, 1379-1386, doi:10.3892/or.2017.5407 (2017).
5 Tumiati, M. et al. A Functional Homologous Recombination Assay Predicts Primary Chemotherapy Response and Long-Term Survival in Ovarian Cancer Patients. Clin Cancer Res 24, 4482-4493, doi:10.1158/1078-0432.CCR-17-3770 (2018).
6 Zhao, E. Y. et al. Homologous Recombination Deficiency and Platinum-Based Therapy Outcomes in Advanced Breast Cancer. Clin Cancer Res 23, 7521-7530, doi:10.1158/1078-0432.CCR-17-1941 (2017).
7 Farmer, H. et al. Targeting the DNA repair defect in BRCA mutant cells as a therapeutic strategy. Nature 434, 917-921, doi:10.1038/nature03445 (2005).
8 Bryant, H. E. et al. Specific killing of BRCA2-deficient tumours with inhibitors of poly(ADP-ribose) polymerase. Nature 434, 913-917, doi:10.1038/nature03443 (2005).
9 El-Khamisy, S. F., Masutani, M., Suzuki, H. & Caldecott, K. W. A requirement for PARP-1 for the assembly or stability of XRCC1 nuclear foci at sites of oxidative DNA damage. Nucleic Acids Res 31, 5526-5533, doi:10.1093/nar/gkg761 (2003).
10 Murai, J. et al. Trapping of PARP1 and PARP2 by Clinical PARP Inhibitors. Cancer research 72, 5588-5599, doi:10.1158/0008-5472.CAN-12-2753 (2012).
11 Murai, J. et al. Stereospecific PARP trapping by BMN 673 and comparison with olaparib and rucaparib. Mol Cancer Ther 13, 433-443, doi:10.1158/1535-7163.MCT-13-0803 (2014).
12 Patel, A. G., Sarkaria, J. N. & Kaufmann, S. H. Nonhomologous end joining drives poly(ADP-ribose) polymerase (PARP) inhibitor lethality in homologous recombination-deficient cells. Proceedings of the National Academy of Sciences of the United States of America 108, 3406-3411, doi:10.1073/pnas.1013715108 (2011).
13 Dev, H. et al. Shieldin complex promotes DNA end-joining and counters homologous recombination in BRCA1-null cells. Nat Cell Biol 20, 954-965, doi:10.1038/s41556-018-0140-1 (2018).
14 Tobalina, L., Armenia, J., Irving, E., O'Connor, M. J. & Forment, J. V. A meta-analysis of reversion mutations in BRCA genes identifies signatures of DNA end-joining repair mechanisms driving therapy resistance. Ann Oncol, doi:10.1016/j.annonc.2020.10.470 (2020).
15 Dent, R. A. et al. Phase I trial of the oral PARP inhibitor olaparib in combination with paclitaxel for first- or second-line treatment of patients with metastatic triple-negative breast cancer. Breast Cancer Res 15, R88, doi:10.1186/bcr3484 (2013).
16 Fong, P. C. et al. Inhibition of poly(ADP-ribose) polymerase in tumors from BRCA mutation carriers. N Engl J Med 361, 123-134, doi:10.1056/NEJMoa0900212 (2009). Sandhu, S. K. et al. The poly(ADP-ribose) polymerase inhibitor niraparib (MK4827) in
17 BRCA mutation carriers and patients with sporadic cancer: a phase 1 dose-escalation trial. Lancet Oncol 14, 882-892, doi:10.1016/S1470-2045(13)70240-7 (2013).
18 Lord, C. J. & Ashworth, A. PARP inhibitors: Synthetic lethality in the clinic. Science 355, 1152-1158, doi:10.1126/science.aam7344 (2017).
19 Waks, A. G. et al. Reversion and non-reversion mechanisms of resistance to PARP inhibitor or platinum chemotherapy in BRCA1/2-mutant metastatic breast cancer. Ann Oncol, doi:10.1016/j.annonc.2020.02.008 (2020).
20 Banda, K., Swisher, E. M., Wu, D., Pritchard, C. C. & Gadi, V. K. Somatic Reversion of Germline BRCA2 Mutation Confers Resistance to Poly(ADP-ribose) Polymerase Inhibitor Therapy. JCO Precision Oncology, 1-6, doi:10.1200/po.17.00044 (2018). Carneiro, B. A. et al. Acquired Resistance to Poly (ADP-ribose) Polymerase Inhibitor
21 Olaparib in BRCA2-Associated Prostate Cancer Resulting From Biallelic BRCA2 Reversion Mutations Restores Both Germline and Somatic Loss-of-Function Mutations. JCO Precision Oncology, 1-8, doi:10.1200/po.17.00176 (2018).
22 Cheng, H. H., Salipante, S. J., Nelson, P. S., Montgomery, B. & Pritchard, C. C. Polyclonal BRCA2 Reversion Mutations Detected in Circulating Tumor DNA After Platinum Chemotherapy in a Patient With Metastatic Prostate Cancer. JCO Precision Oncology, 1-5, doi:10.1200/po.17.00169 (2018).
23 Pettitt, S. J. et al. Clinical BRCA1/2 Reversion Analysis Identifies Hotspot Mutations and Predicted Neoantigens Associated with Therapy Resistance. Cancer Discov 10, 1475-1488, doi:10.1158/2159-8290.CD-19-1485 (2020).
24 Lin, K. K. et al. BRCA Reversion Mutations in Circulating Tumor DNA Predict Primary and Acquired Resistance to the PARP Inhibitor Rucaparib in High-Grade Ovarian Carcinoma. Cancer Discov 9, 210-219, doi:10.1158/2159-8290.CD-18-0715 (2019).
25 Gornstein, E. L. et al. BRCA2 Reversion Mutation Associated With Acquired Resistance to Olaparib in Estrogen Receptor-positive Breast Cancer Detected by Genomic Profiling of Tissue and Liquid Biopsy. Clin Breast Cancer 18, 184-188, doi:10.1016/j.clbc.2017.12.010 (2018).
26 Afghahi, A. et al. Tumor BRCA1 Reversion Mutation Arising during Neoadjuvant Platinum-Based Chemotherapy in Triple-Negative Breast Cancer Is Associated with Therapy Resistance. Clin Cancer Res 23, 3365-3370, doi:10.1158/1078-0432.CCR-16-2174 (2017).
27 Loibl, S. et al. Addition of the PARP inhibitor veliparib plus carboplatin or carboplatin alone to standard neoadjuvant chemotherapy in triple-negative breast cancer (BrighTNess): a randomised, phase 3 trial. Lancet Oncol 19, 497-509, doi:10.1016/S1470-2045(18)30111-6 (2018).
28 Vidula, N. et al. Routine plasma-based genotyping to comprehensively detect germline, somatic, and reversion BRCA mutations among patients with advanced solid tumors. Clin Cancer Res, doi:10.1158/1078-0432.CCR-19-2933 (2020).
29 Parikh, A. R. et al. Liquid versus tissue biopsy for detecting acquired resistance and tumor heterogeneity in gastrointestinal cancers. Nature medicine 25, 1415-1421, doi:10.1038/s41591-019-0561-9 (2019).
30 McGranahan, N. et al. Clonal status of actionable driver events and the timing of mutational processes in cancer evolution. Sci Transl Med 7, 283ra254, doi:10.1126/scitranslmed.aaa1408 (2015).
31 Pinto, C. et al. Recommendations for the implementation of BRCA testing in the care and treatment pathways of ovarian cancer patients. Future Oncol 12, 2071-2075, doi:10.2217/fon-2016-0189 (2016).
32 Beaubier, N. et al. Integrated genomic profiling expands clinical options for patients with cancer. Nat Biotechnol 37, 1351-1360, doi:10.1038/s41587-019-0259-z (2019).
33 Beaubier, N. et al. Clinical validation of the tempus xT next-generation targeted oncology sequencing assay. Oncotarget 10, 2384-2396, doi:10.18632/oncotarget.26797 (2019).
34 Robinson, J. T. et al. Integrative genomics viewer. Nat Biotechnol 29, 24-26, doi:10.1038/nbt.1754 (2011).

What is claimed:

1. A method of treating a subject that has been diagnosed with cancer, the method comprising:
a. at a first time point:
(1) obtaining at least three biological samples from the subject, wherein at least one of the samples comprises a solid tumor sample, wherein at least one of the samples comprises a matched normal sample, and wherein at least one of the samples comprises a blood plasma sample;
(2) isolating nucleic acid from each sample;
(3) sequencing the nucleic acid from each of the samples to obtain genetic sequence information;
(4) comparing the sequence information obtained in (3) to a wild-type reference sequence for the species of the subject to identify mutations; and
(5) treating the subject with a first cancer treatment based on the comparison made in (4);
b. at a second time point, obtaining at least one biological sample from the subject, wherein the at least one biological sample comprises a solid tumor sample, a blood plasma sample, or both a solid tumor sample and a blood plasma sample, and repeating steps (2)-(3);
c. comparing the sequence information obtained in step (b)(3) at the second time point with the sequence information obtained in step (a)(3) at the first time point; and
d. treating the subject with a second cancer treatment based on the comparison made in step (c).

2. The method of claim 1, wherein a mutation that was not detected at the first time point is detected at the second time point in the solid tumor sample, in the blood plasma sample, or in both the solid tumor sample and the blood plasma sample.

3. The method of claim 1, wherein a reversion mutation is detected at the second time point in the solid tumor sample, in the blood plasma sample, or in both the solid tumor sample and the blood plasma sample.

4. The method of claim 1, wherein the sequence information obtained from the solid tumor and the blood plasma sample is the same at the first time point but is different at the second time point, and wherein the subject is subsequently evaluated for metastases.

5. The method of claim 1, wherein the second time point is taken after disease progression occurs.

6. The method of claim 5, wherein the cancer has relapsed, progressed, metastasized, or developed resistance to the first cancer treatment.

7. The method of claim 5, wherein the second time point is taken at or near the end of the first cancer treatment.

8. The method of claim 5, wherein the cancer is breast cancer, ovarian cancer, prostate cancer, pancreatic cancer, or melanoma.

9. The method of claim 1, wherein a mutation in the APC, ATM, AXIN2, BMPRIA, BRCA1, BRCA2, BRIP1, CDC73, CDH1, CDK4, CDKN2A, CEBPA, CHEK2, DKC1, EGFR, EPCAM ETV6, FH, FLCN, GATA2, GREMJ, KIT, MAX, MEN1, MET, MLH1, MSH2, MSH3, MSH6, MUTYH, NBN, NF1, NF2, NTHL1, PALB2, PDGFRA, PMS2, POLD1, POLE, PRKAR1A, PTCH1, PTEN, RAD51C, RAD51D, RB1, RET, RUNX1, SCG5, SDHAF2, SDHB, SDHC, SDHD, SMAD4, STK11, TERC, TINF2, TP53, TSC1, TSC2, VHL, or WT1 gene is detected in the solid tumor sample at the first time point.

10. The method of claim 9, wherein the same mutation is detected in the matched normal sample at the first time point.

11. The method of claim 1, wherein the first cancer treatment is discontinued based on the comparison made in step (c), and wherein the second cancer treatment is different than the first cancer treatment.

12. The method of claim 1, wherein nucleic acid isolated from the blood plasma sample comprises circulating tumor DNA.

13. The method of claim 1, wherein the nucleic acid isolated from the solid tumor sample comprises DNA, and wherein the DNA is sequenced using whole genome sequencing.

14. The method of claim 1, wherein the nucleic acid isolated from the solid tumor sample comprises RNA, and wherein the RNA is sequenced using whole transcriptome sequencing.

15. The method of claim 1, wherein the subject is a human.

16. The method of claim 1, wherein the first cancer treatment is a drug against which resistance mechanisms are known.

17. The method of claim 16, wherein the first cancer treatment is a PARP inhibitor or a platinum-based therapy.

18. The method of claim 1 further comprising:
e. at an Nth time point, repeating steps (1)-(3);
f. comparing the sequence information obtained for the solid tumor sample in step (e)(3) at the Nth time point with the sequence information obtained for the solid tumor sample in a corresponding step at an N-1 time point, and comparing the sequence information obtained for the blood plasma sample in step (e)(3) at the Nth time point with the sequence information obtained for the blood plasma sample in the corresponding step at the N-1 time point to identify changes in the sequencing information; and
g. treating the subject with a cancer treatment based on the comparison made in step (f).

19. The method of claim 1, wherein the first and second cancer treatment are different.

20. The method of claim 1, wherein the first and second cancer treatment are the same.
